(19) United States
(12) Plant Patent Application Publication
(10) Pub. No.: US 2012/0042423 P1
(43) Pub. Date: Feb. 16, 2012

(54) Phytophthora Nejat-Spina

(50) ;

(75) Inventor: Najmeh (Setareh) Nejat, Los Angeles, CA (US)

(73) Assignee: Najmeh Nejat, Los Angeles, CA (US)

(21) Appl. No.: 12/931,128

(22) Filed: Jan. 24, 2011

(51) Int. Cl. A01H 15/00

(52) U.S. Cl. PLT/394

(57) ABSTRACT

This project is related to the inventor, Najmeh (Setareh) Nejat, Master of Science thesis. During the research, Phytophthora Nejat-Spina was discovered as a new species in the kingdom of Stramenopila, phylum of Oomycota, class of Pythiales, genus of Phytophthora, and belong to the family of Pythiaceae. Phytophthora Nejat-Spina is a plant symptoms, detected as a root rot in sapling of Ziziphus spina-christi, a plant that is growing up in a small town “Kazeroon” in state of Fars in Iran. Phytophthora Nejat-Spina has genome sequence similarities with Phytophthora arecae and Phytophthora palmivora, and has morphological similarities with spices Phytophthora Porri, Phytophthora Sojae, and Phytophthora Megasperma, but the microbiology results showed a lots of differences with them, a comparison charts are also provided in the upcoming pages.

### CHAPTER 1

### Introduction

1.1 Definition of Phytophthora Nejat-Spina

Phytophthora Nejat-Spina is a plant symptoms, detected as a root rot in sapling of Ziziphus spina-christi, a plant that is growing up in a small town “Kazeroon” in state of Fars in Iran. Phytophthora Nejat-Spina was discovered as a new species in the kingdom of Stramenopila, phylum of Oomycota, class of Pythiales, genus of Phytophthora, and belong to the family of Pythiaceae.

1.2 Microbiological Characteristics

Sporangium
papilla
Sporangium papilla is long papilla and Positive and varies from 2.87 to 12 μm.
shape
varying form, ovoid to broadly ellipsoid, ovoid-sphericale. One apex. Caducous sporangia.
size
The length changes from 34 to 58.88 μm and the width changes from 19.62 to 36.37 μm.

Sporangiophore
Sporangiophore is simple sympodium, external proliferation, un-branched, and sometimes botryose clump Sporangiophore.

Chlamydospores
Production
They are terminal Chlamydospores.
size
Varying from 20.92 to 38.58 μm.
wall thickness
There is no data

Sex Organs
This Spices is homotallic. Oogonia are small globose with tapered base. The antheridia are amphigynous and average 10.36 to 16.38 μm. Also anthridia are paragunous and average 7.99 to 9.82 μm with club shaped for paragynous and spherical shapes for amphigynous.

1.3 Special Characteristics

Multi antherudia
it is very unique.

Oospores
Range from 29.42×31.54 μm. The Oospores for this species is aplerotic.

TABLE 1.1
Diseases caused by Phytophthora Nejat-Spina; positive
means occupance and negative means not occupance

| Range of hosts | Disease |
|---|---|
| Malus Domestica | Negative |
| Juglans regia | Negative |
| Prunus Sp | Negative |
| Prunus dulcis | Negative |
| Lycopesicum esculentum | Positive |
| Pistacia vera | Positive |
| Citrus aurantum | Positive |
| Mespilus germanica | Positive |
| Vicia Sativa | Positive |
| Cucurbita pepo | Positive |
| Solanum melongena | Positive |
| Solanum tuberosum (potato) | Positive |
| Cucamis Sativas (Cucamber) | Positive |

1.4 Symptoms Indications in Some Plants

In the table 1.1 the occupance of Phytophthora Nejat-Spina has been investigated and were shown. Sporangiophore is simple sympodium, external proliferation, un-branched, and sometimes botryose clump Sporangiophore.

Sporangium
papilla
Sporangium papilla is long papilla and Positive and varies from 2.87 to 12 μm.
shape
varying form, ovoid to broadly ellipsoid, ovoid-sphericale. One apex. Caducous sporangia.
size
The length changes from 34 to 58.88 μm and the width changes from 19.62 to 36.37 μm.

Picture scale 400 times of real size. Range from 29.42×31.54 μm. The Oospores for this species is aplerotic.

1.5 Comparison

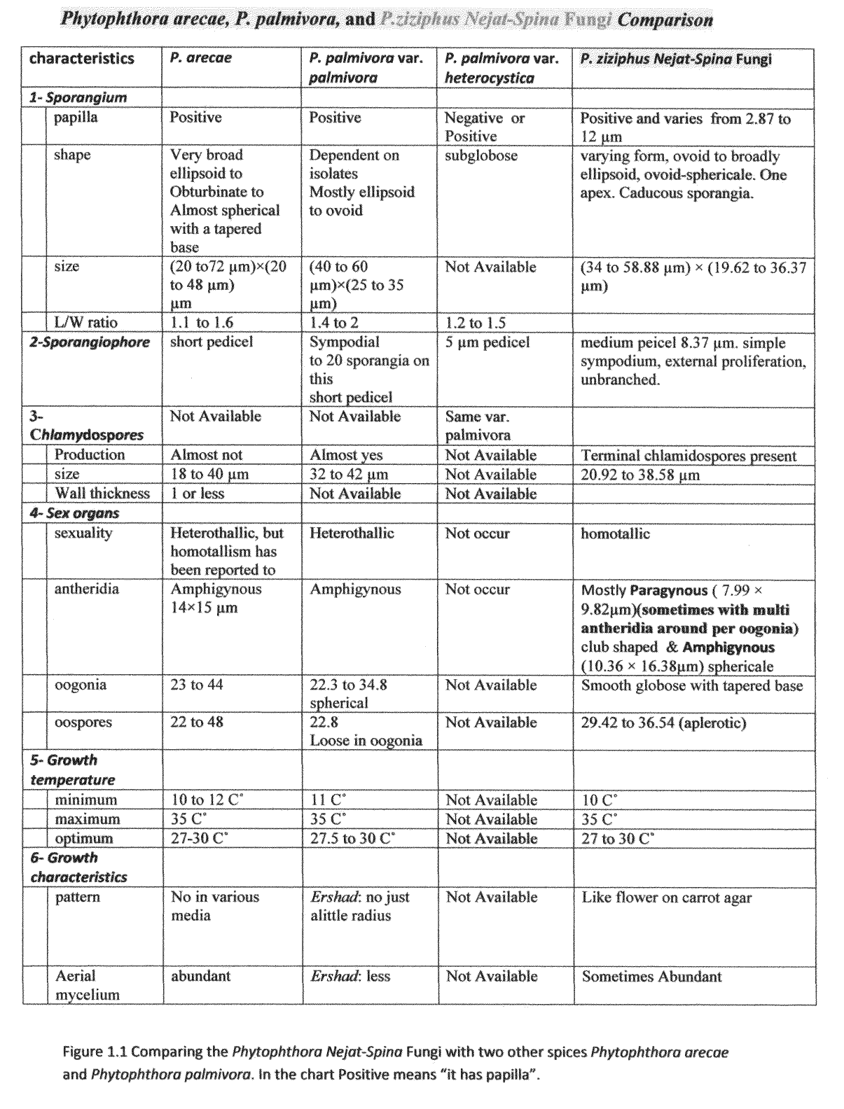

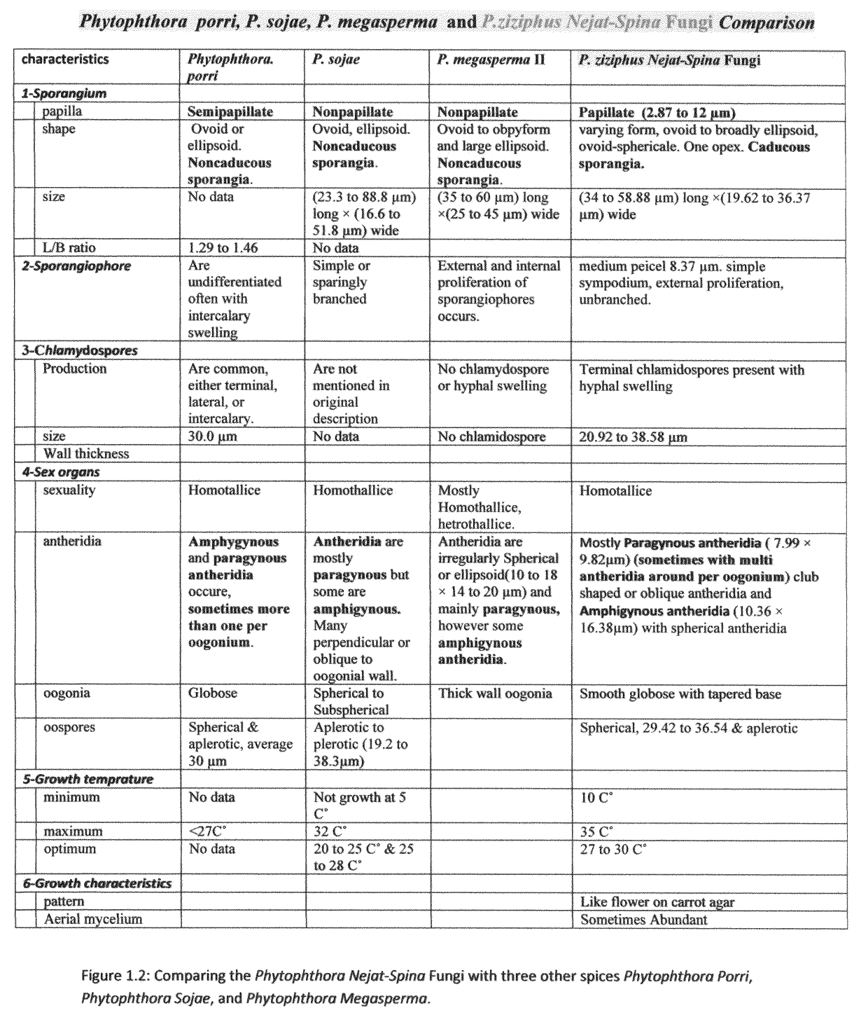

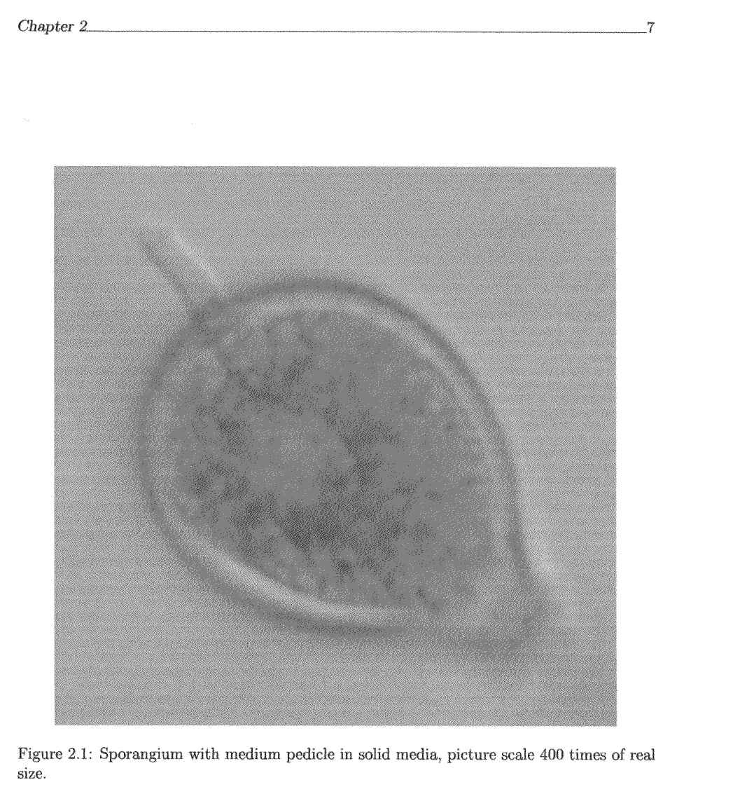

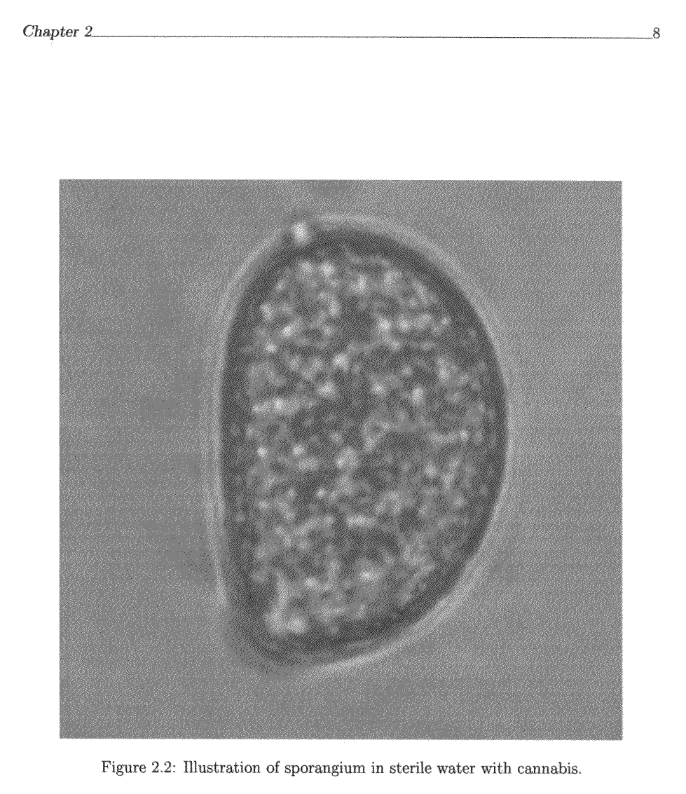

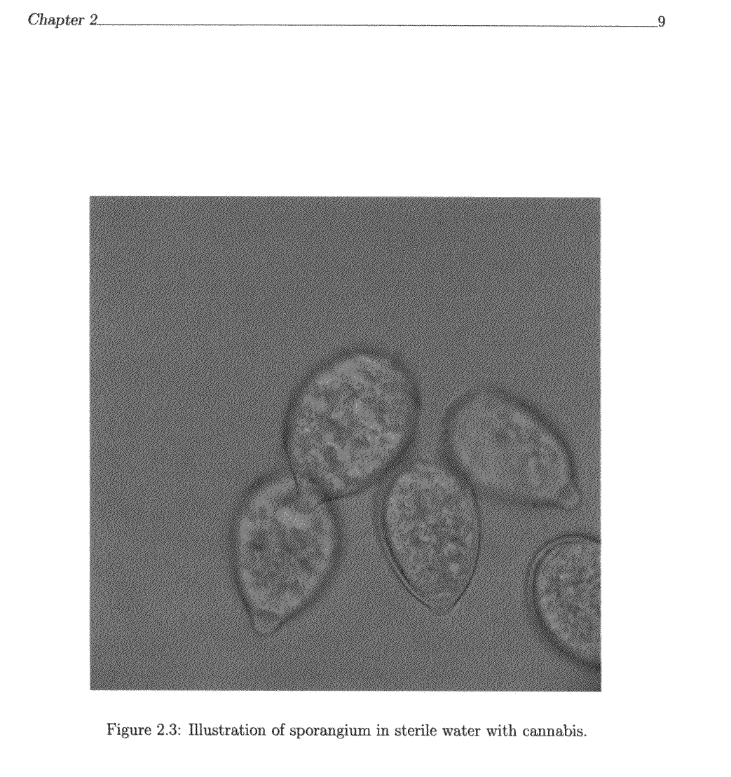

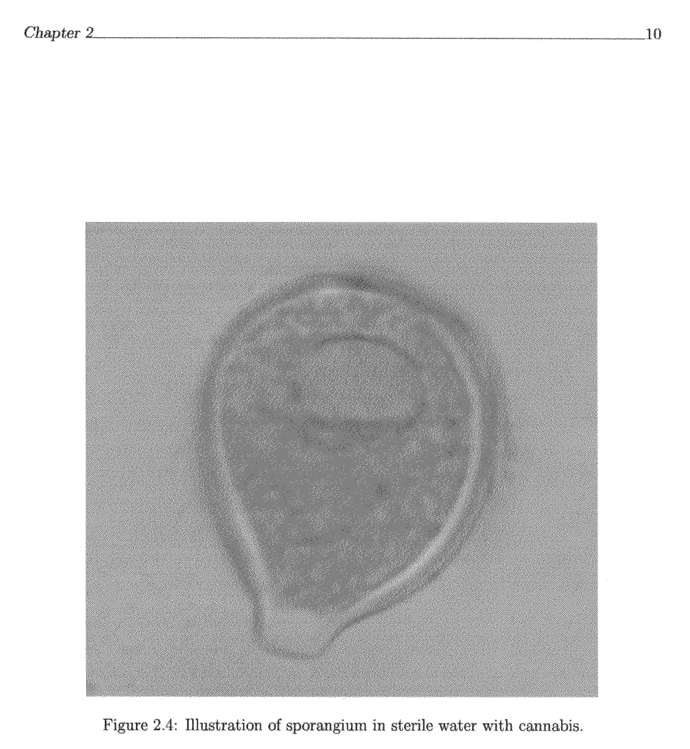

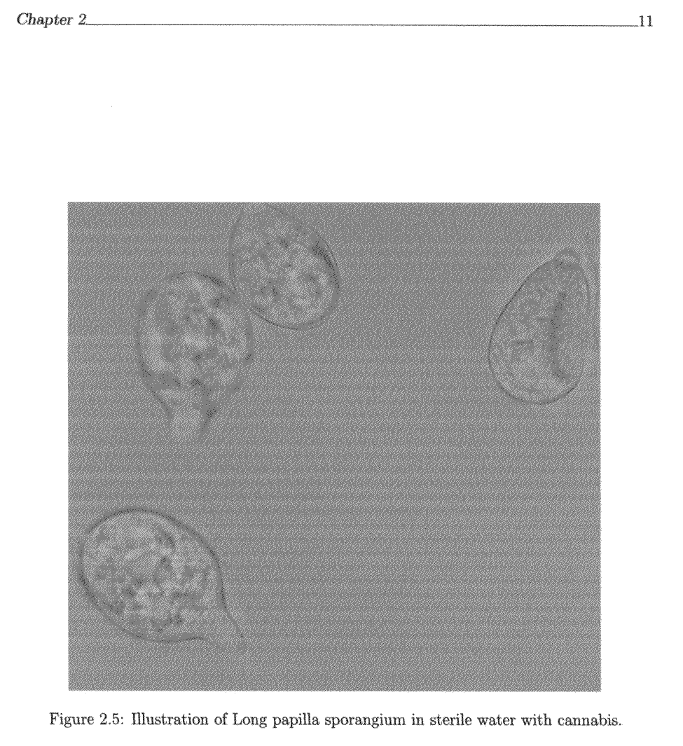

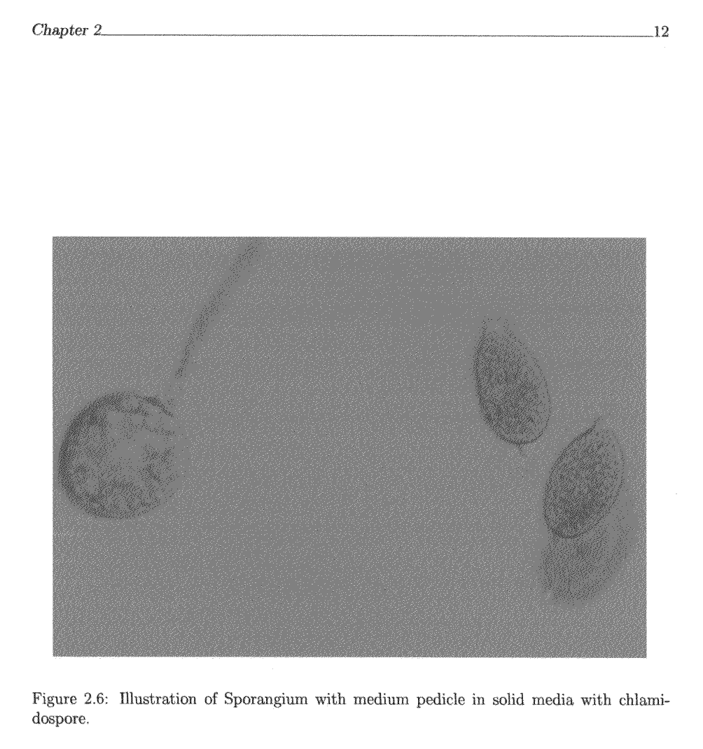

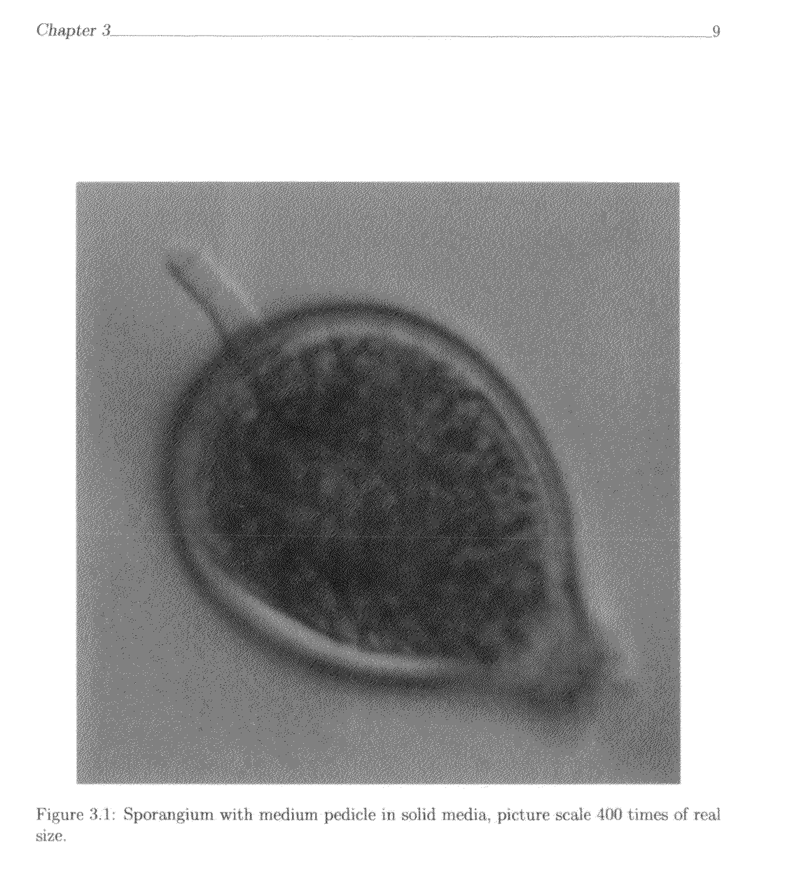

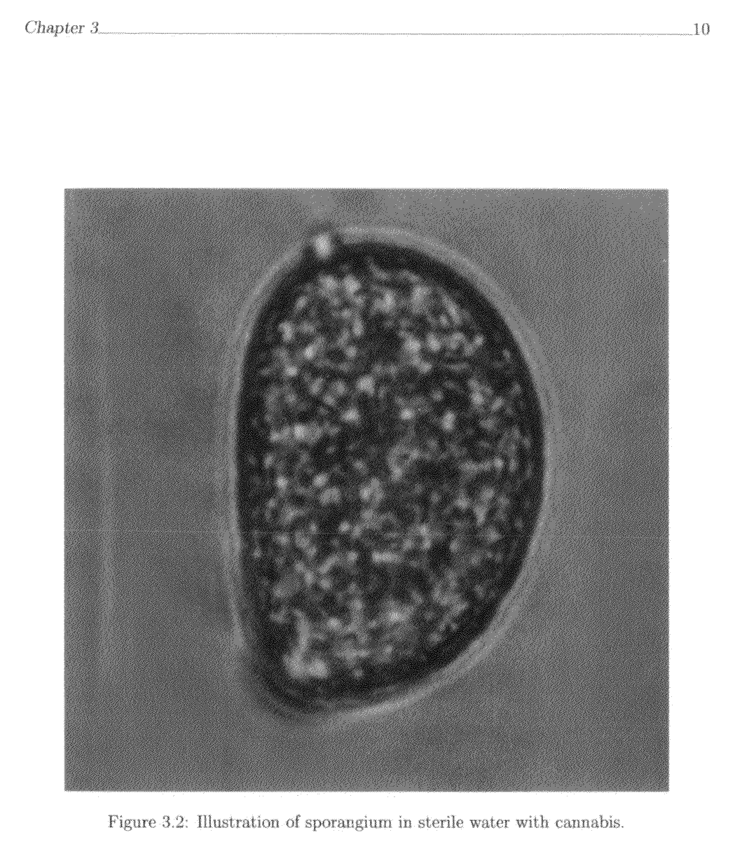

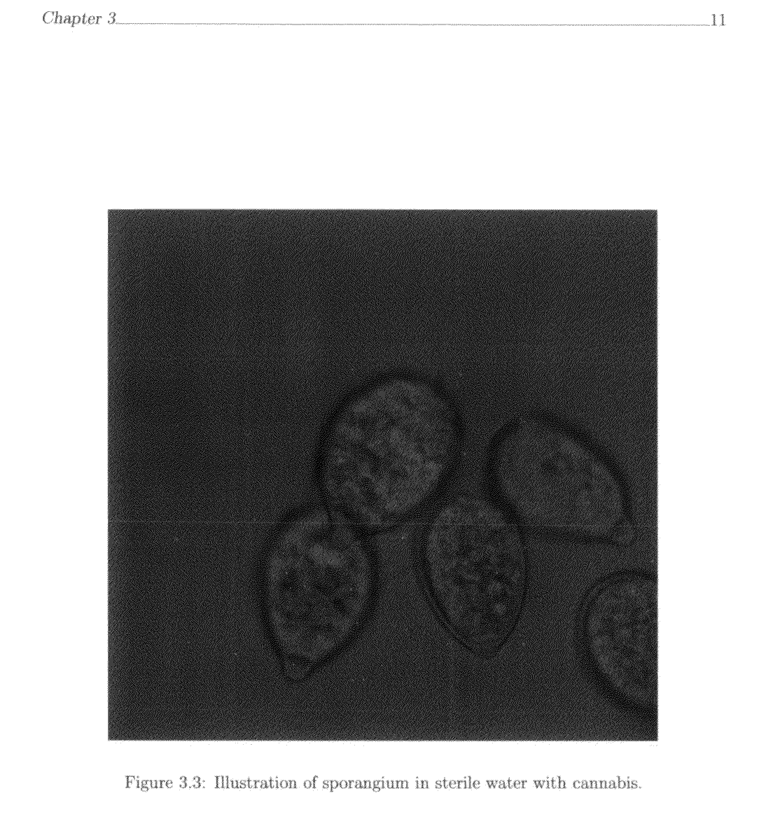

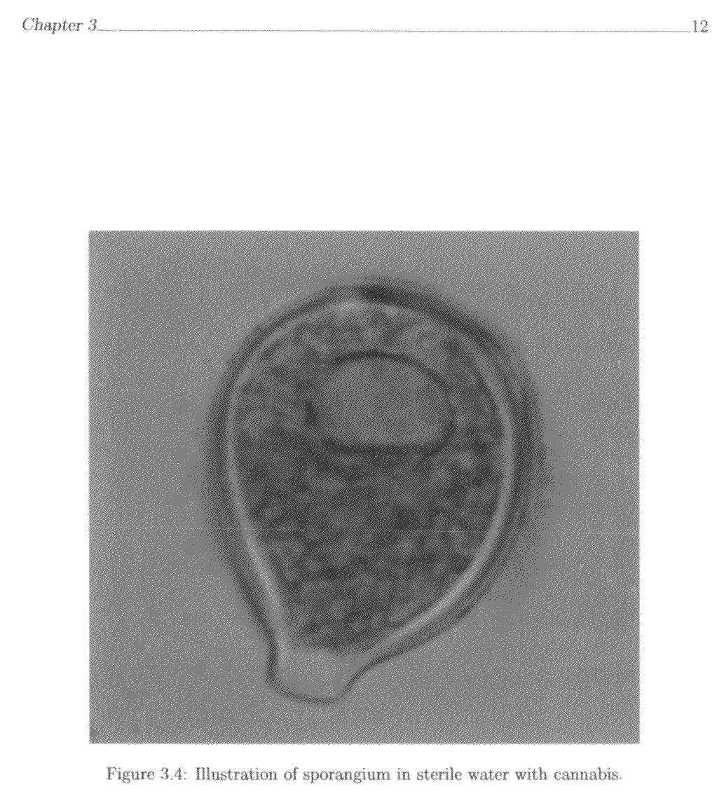

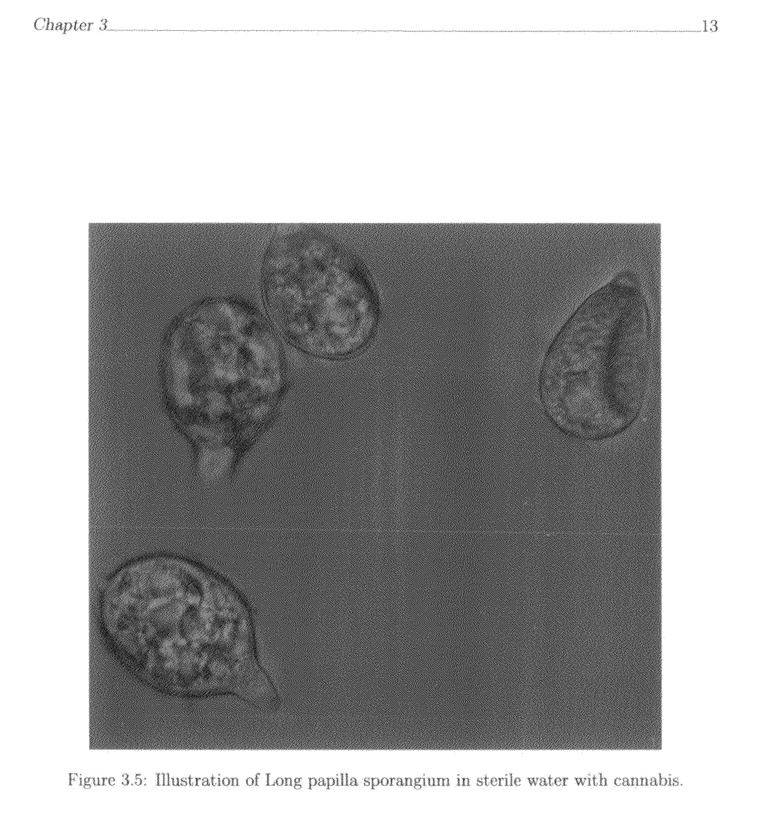

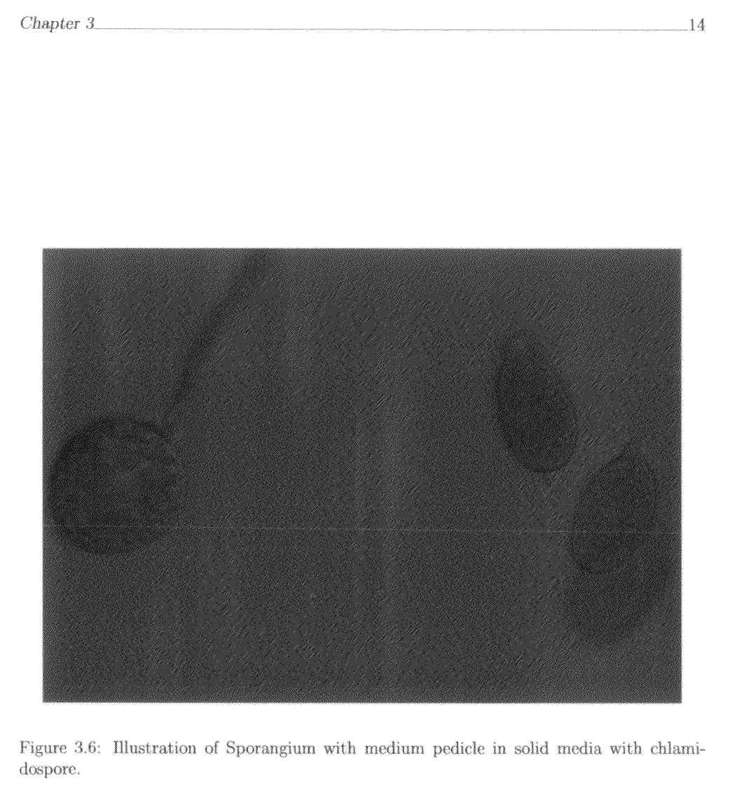

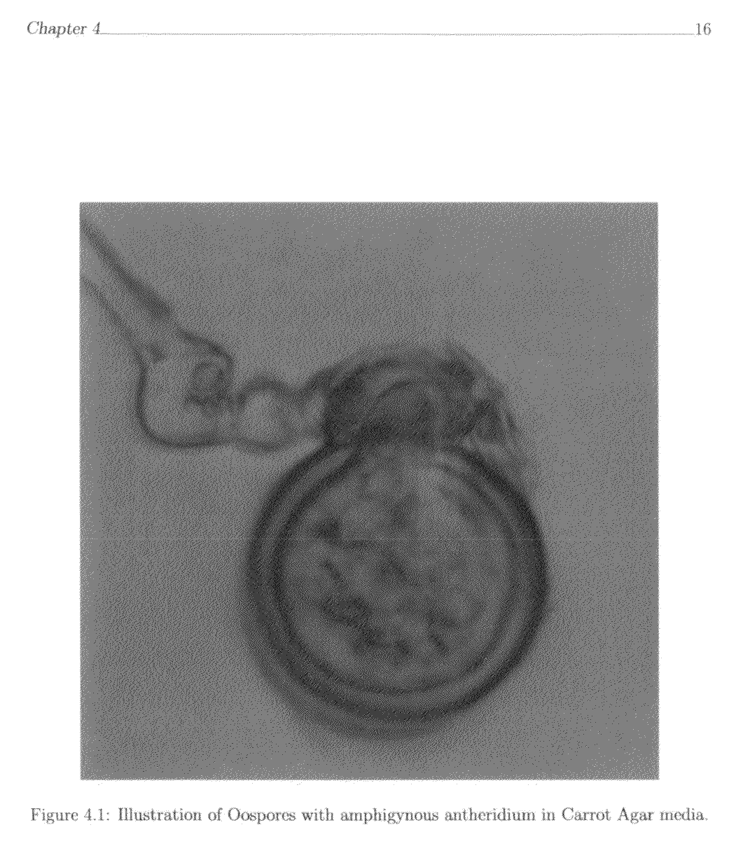

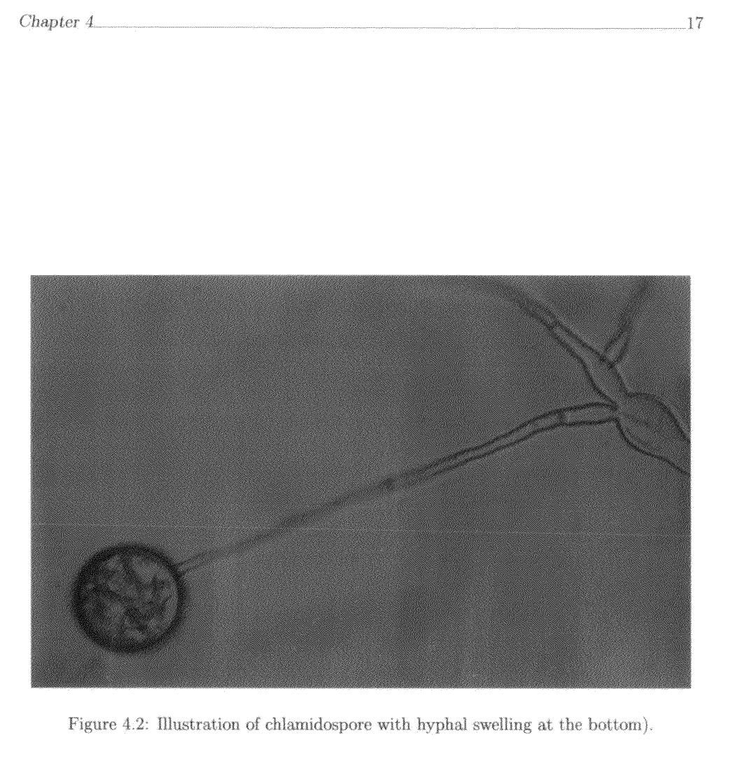

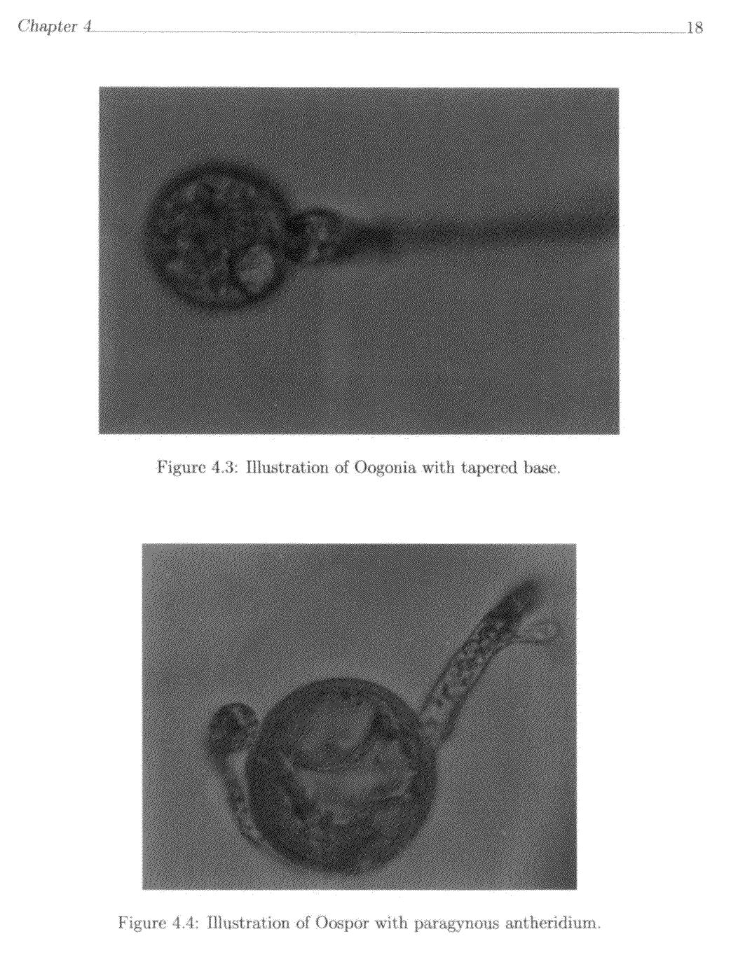

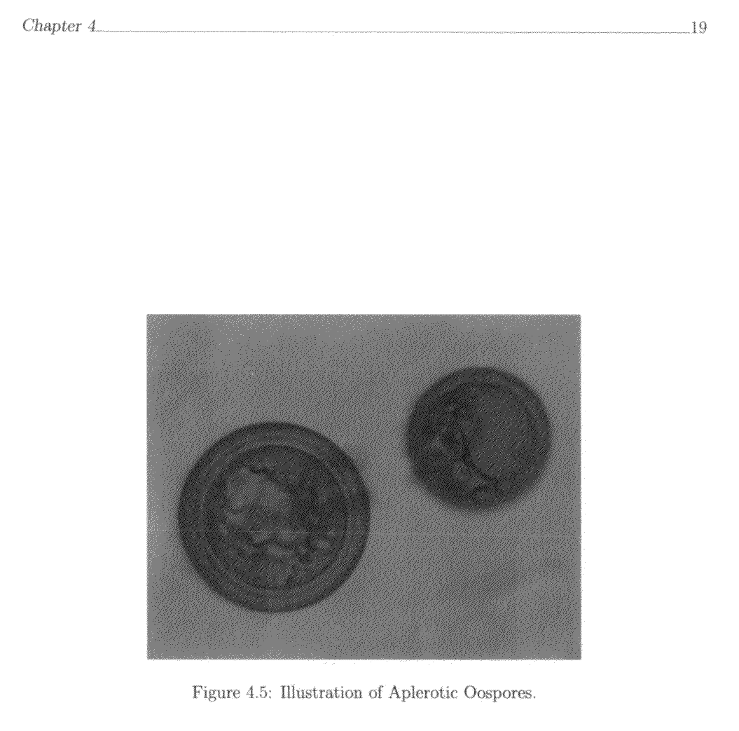

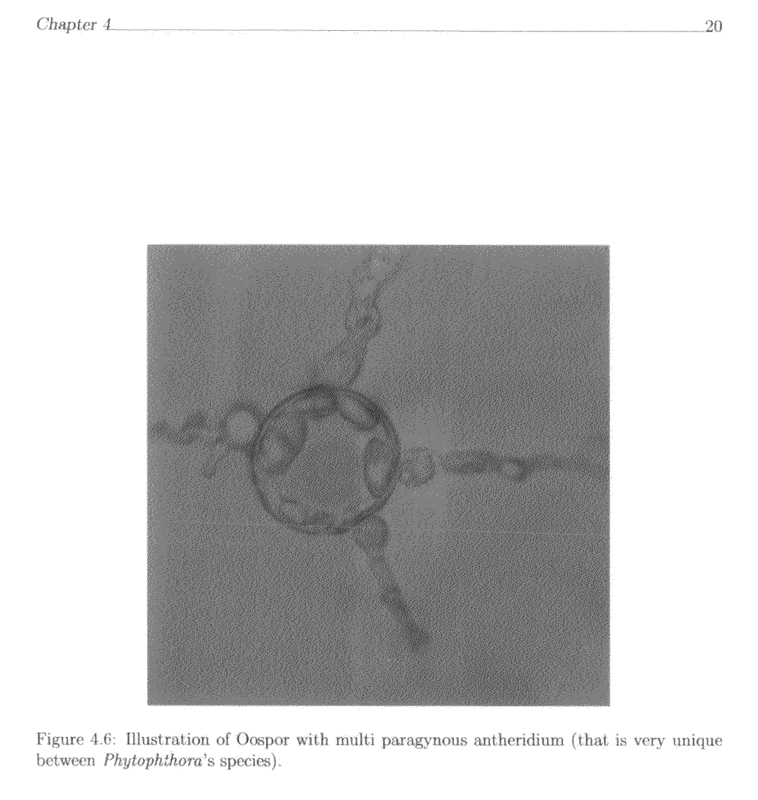

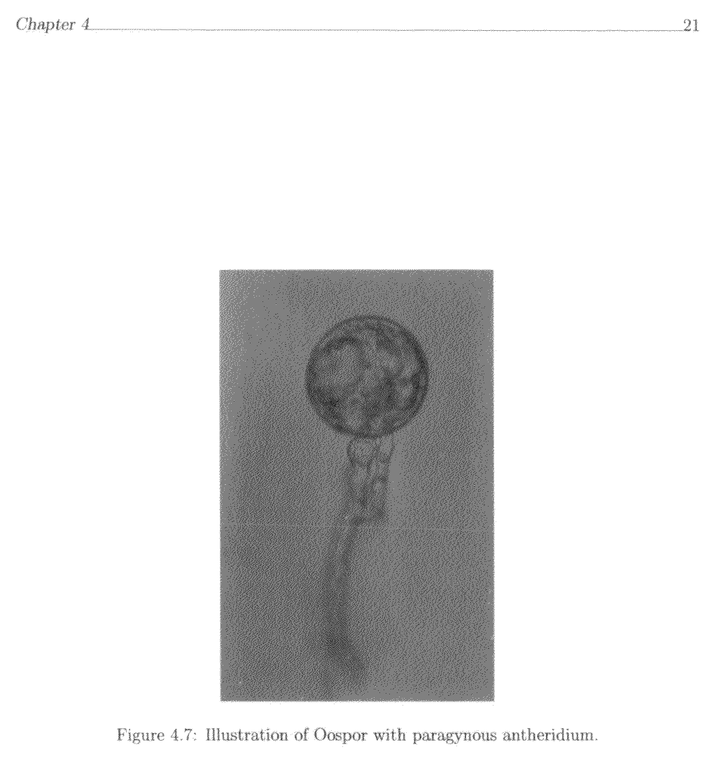

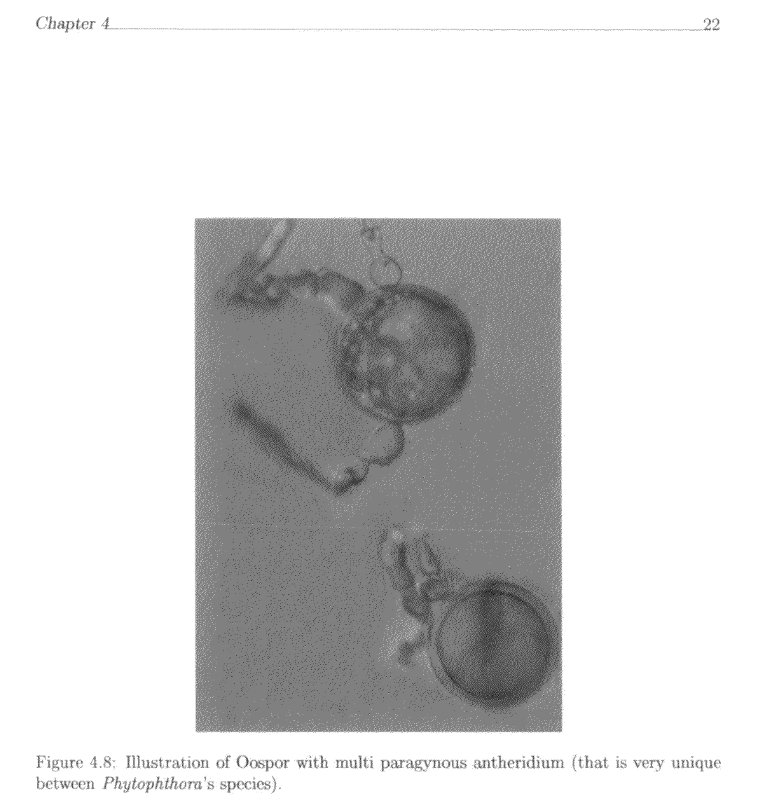

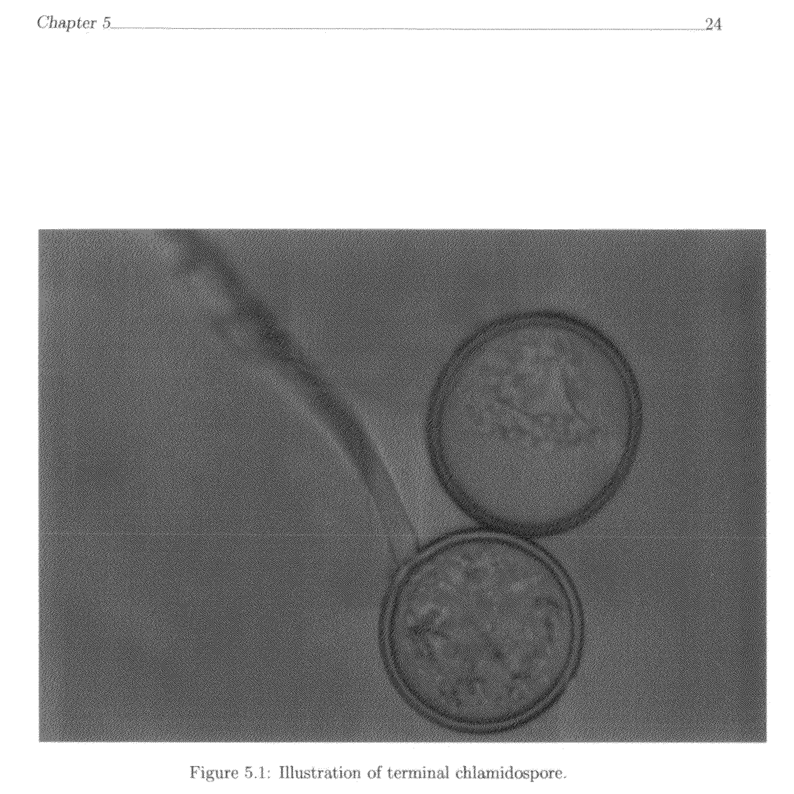

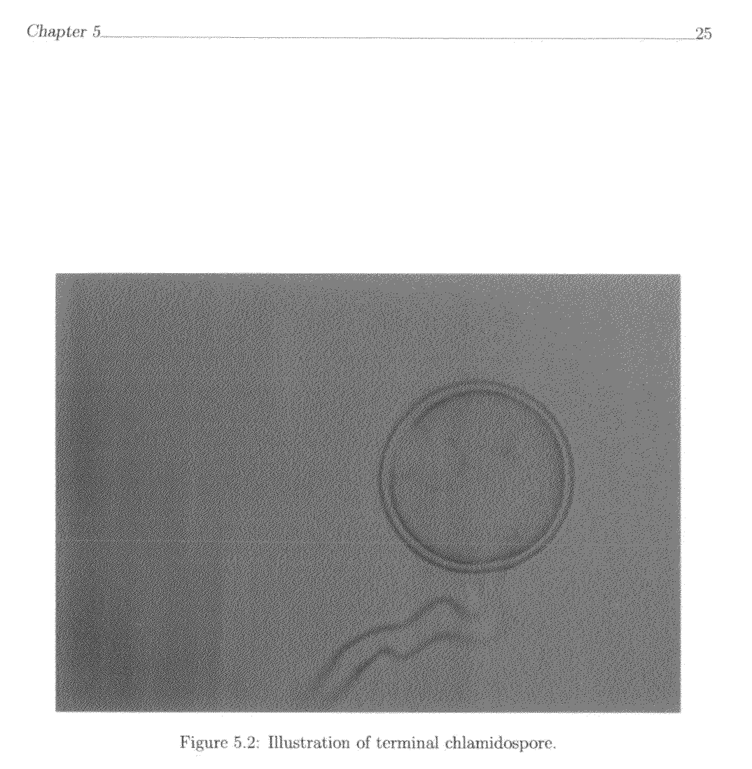

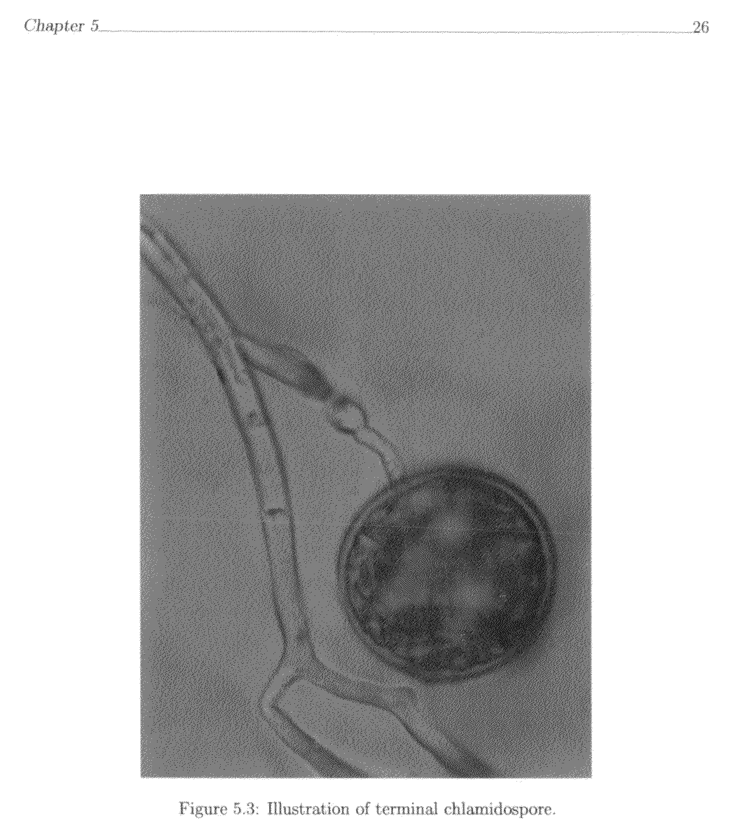

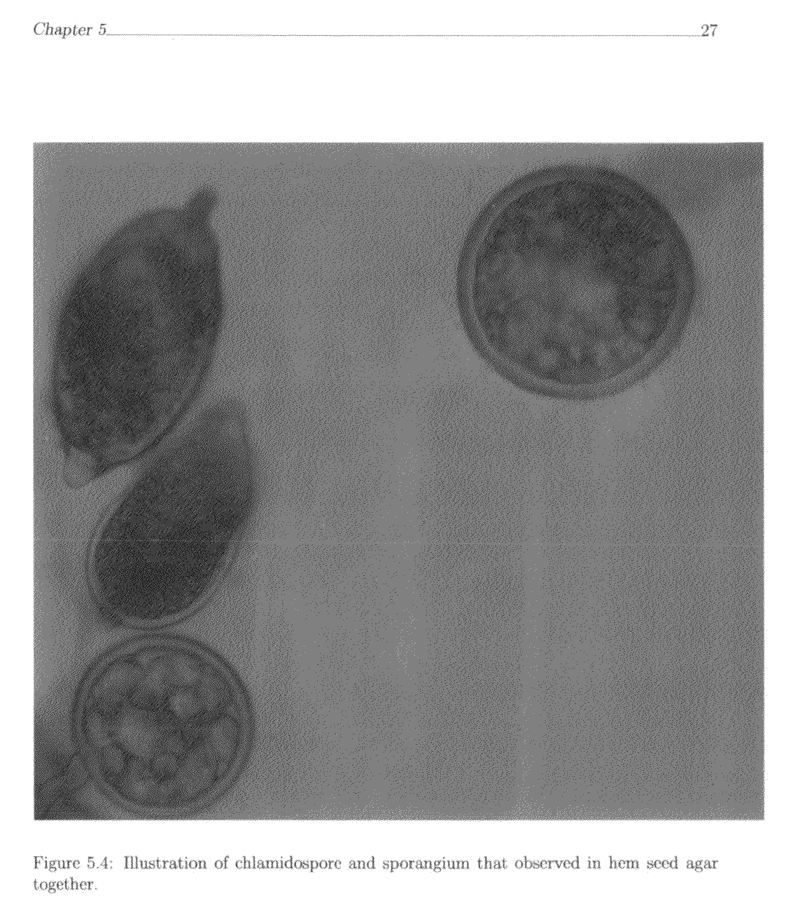

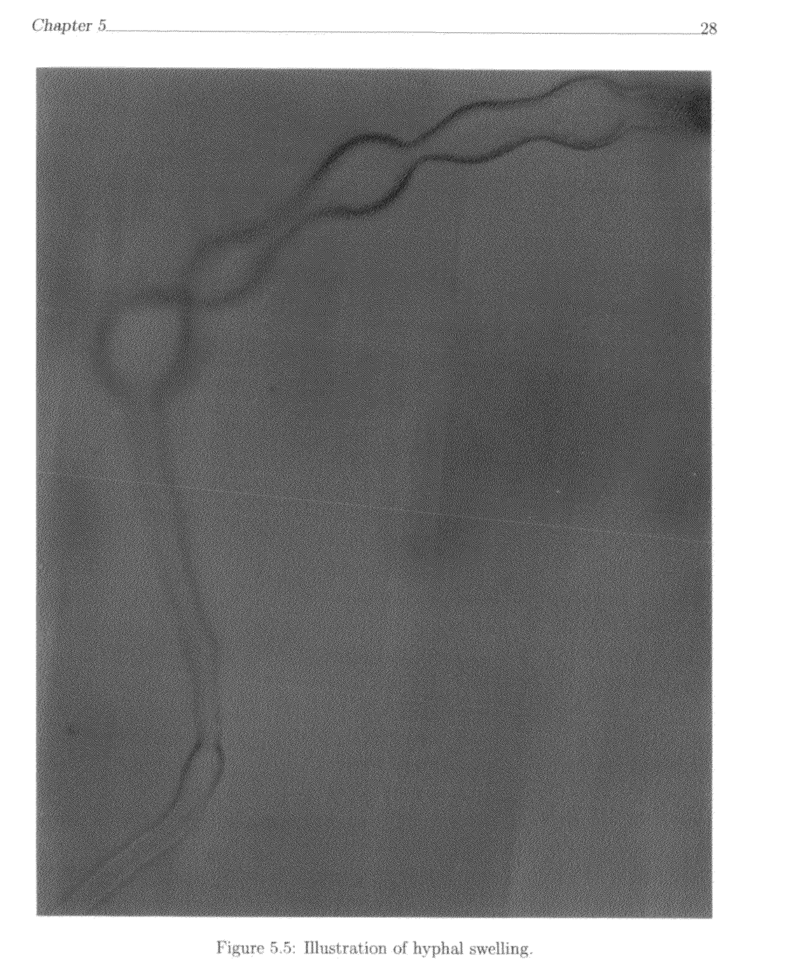

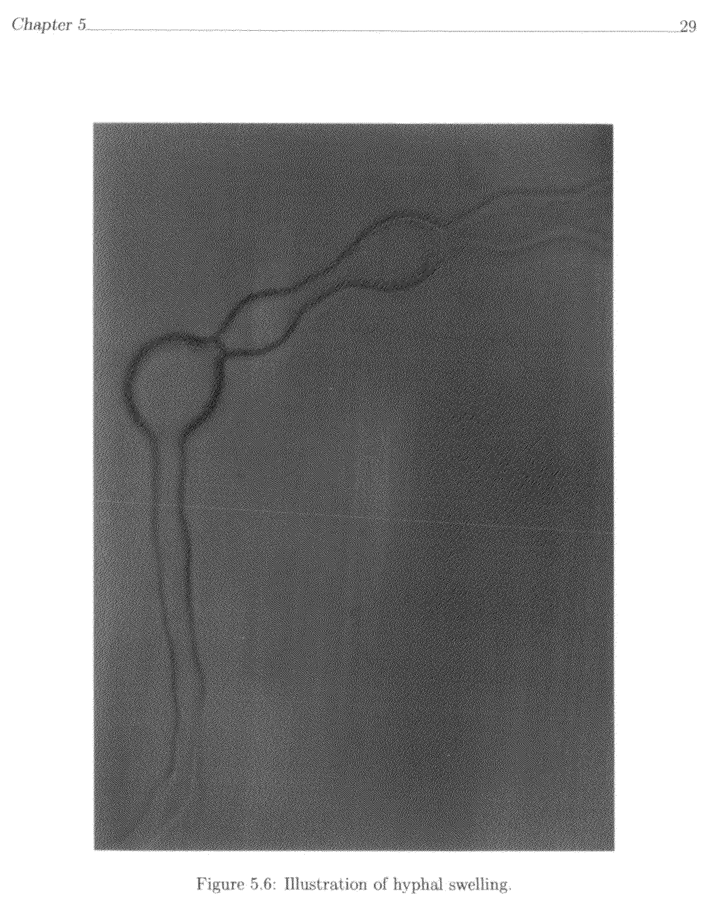

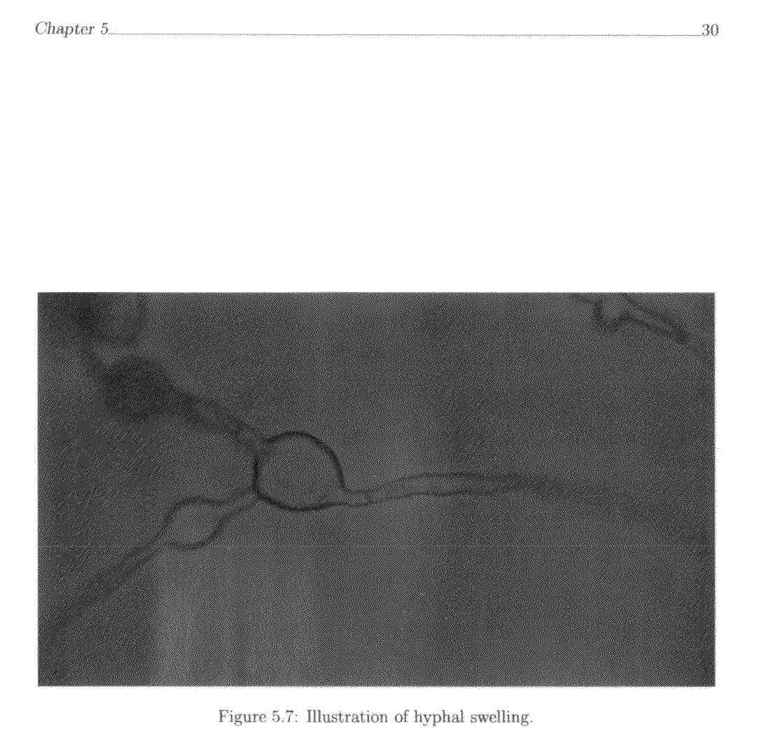

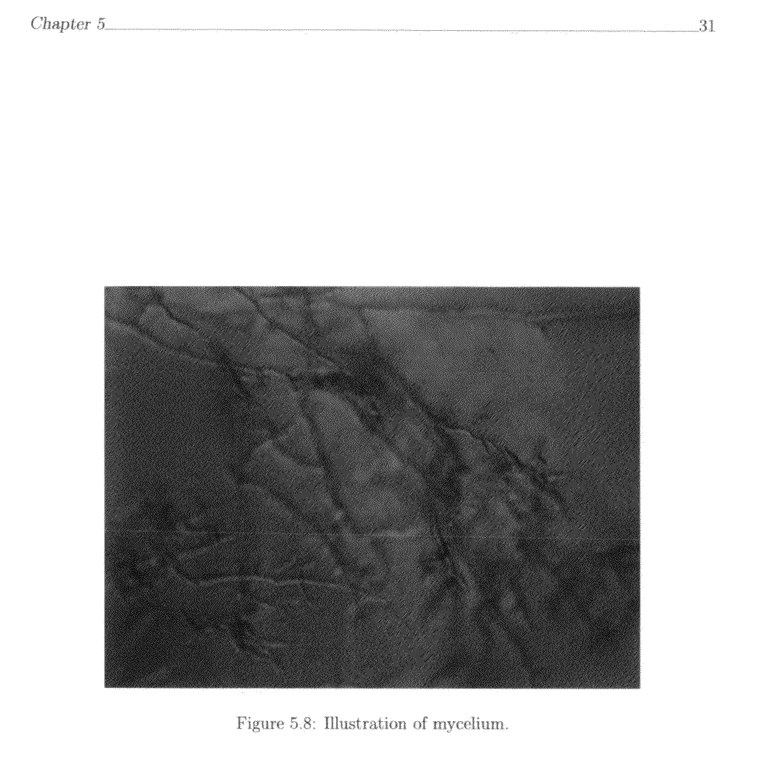

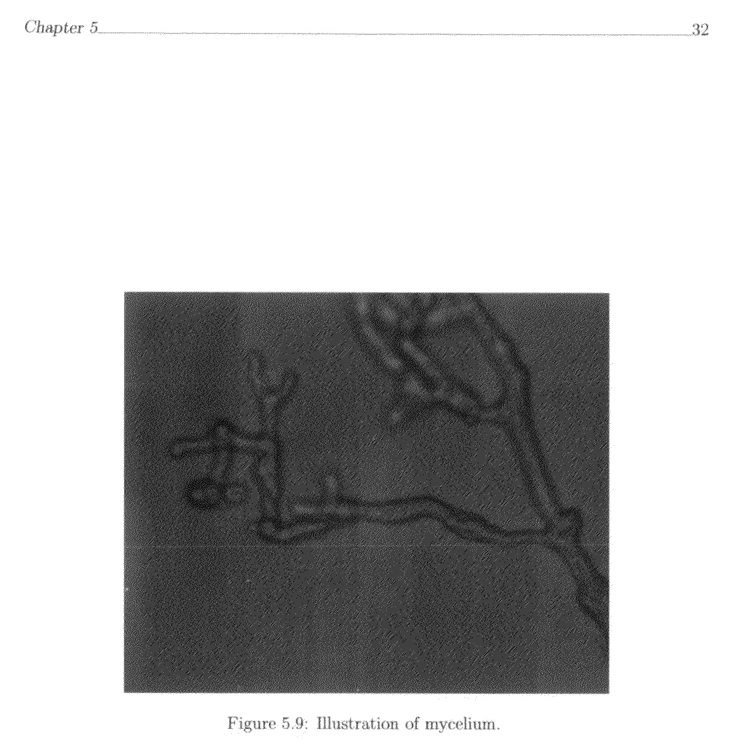

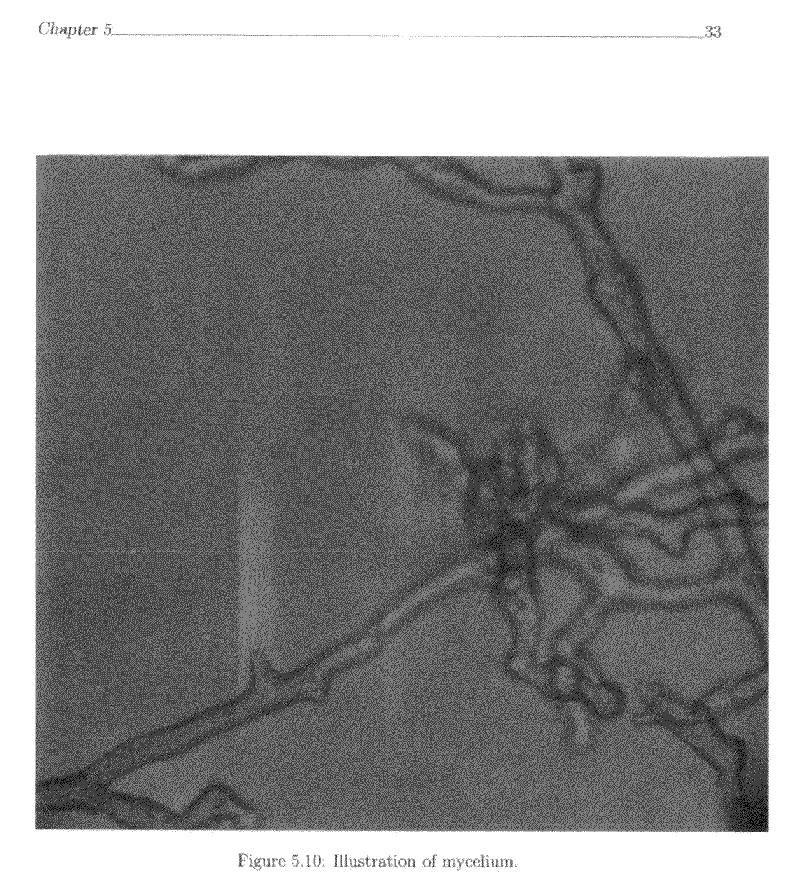

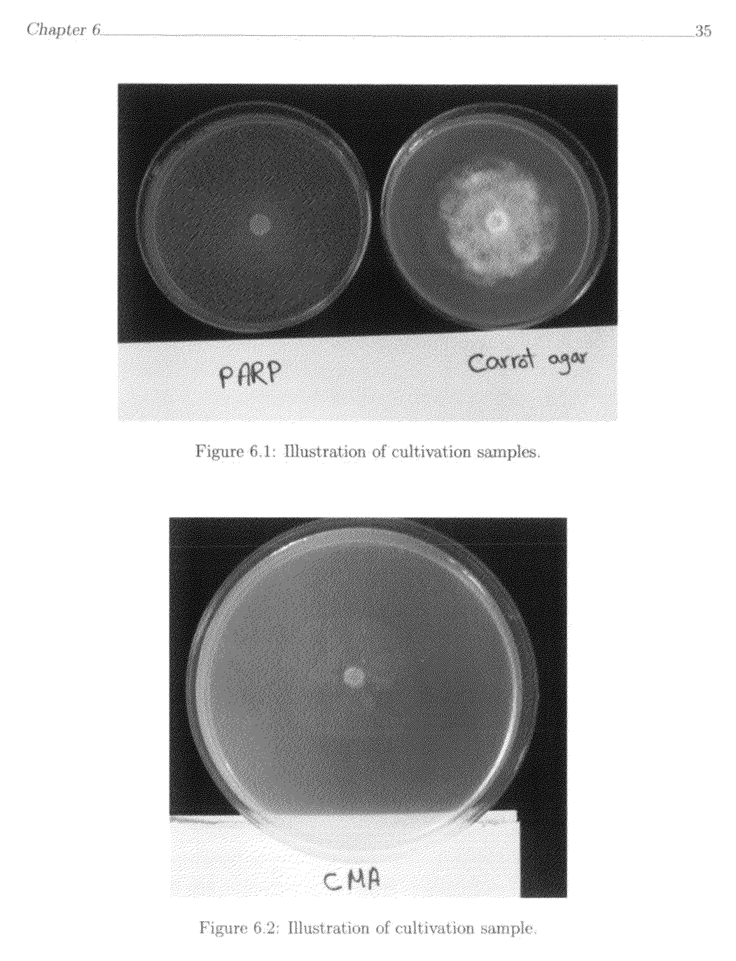

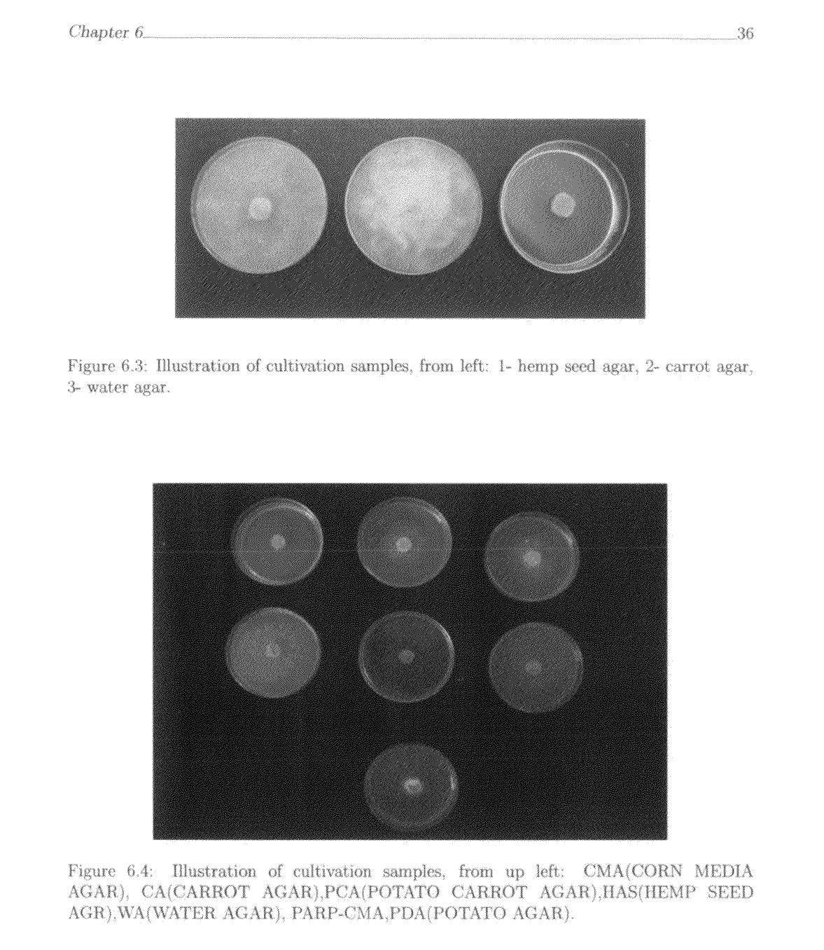

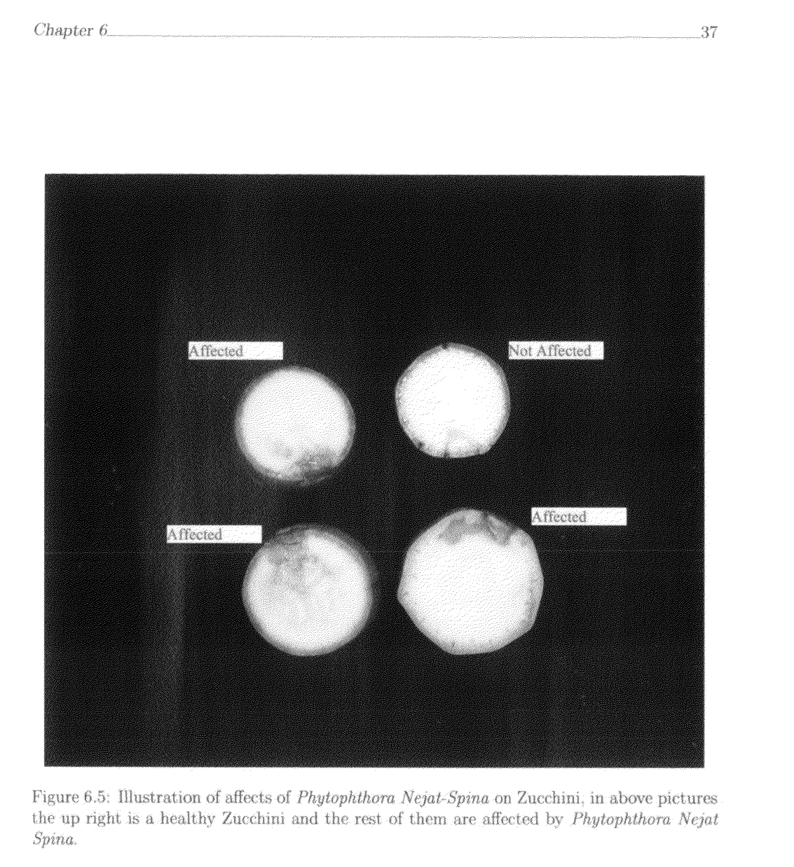

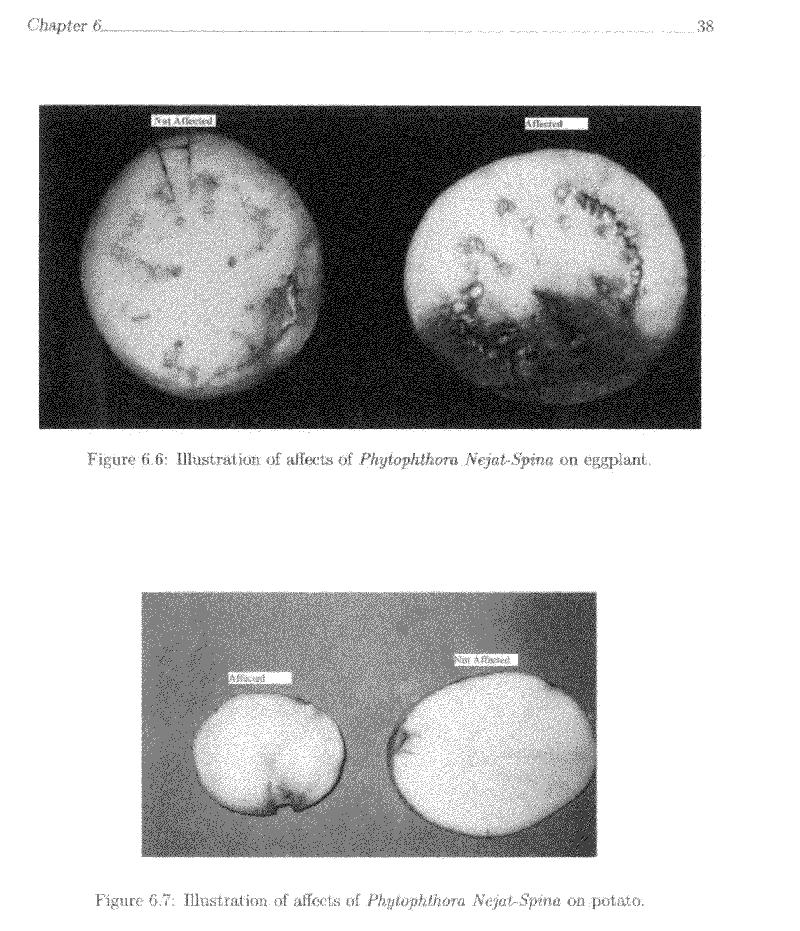

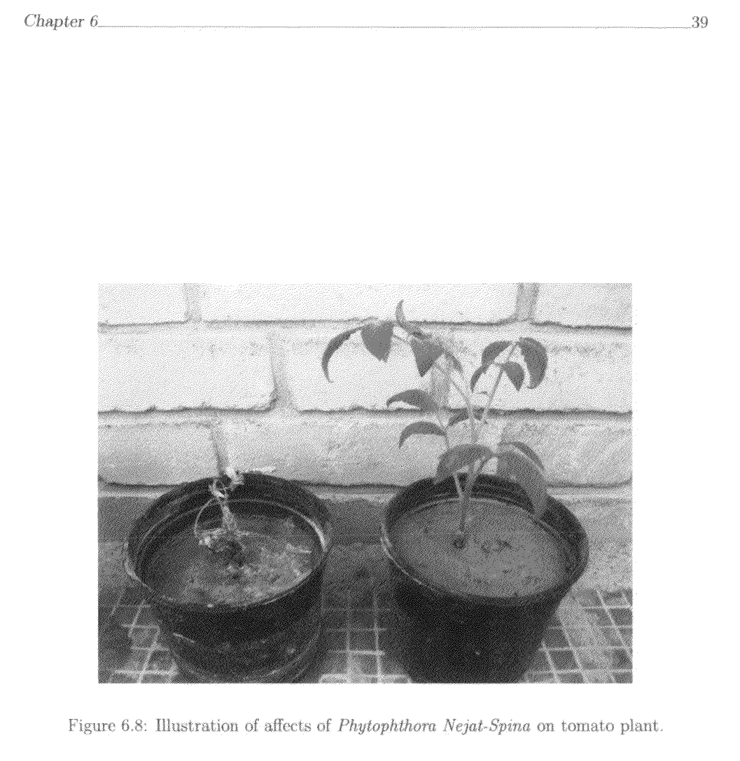

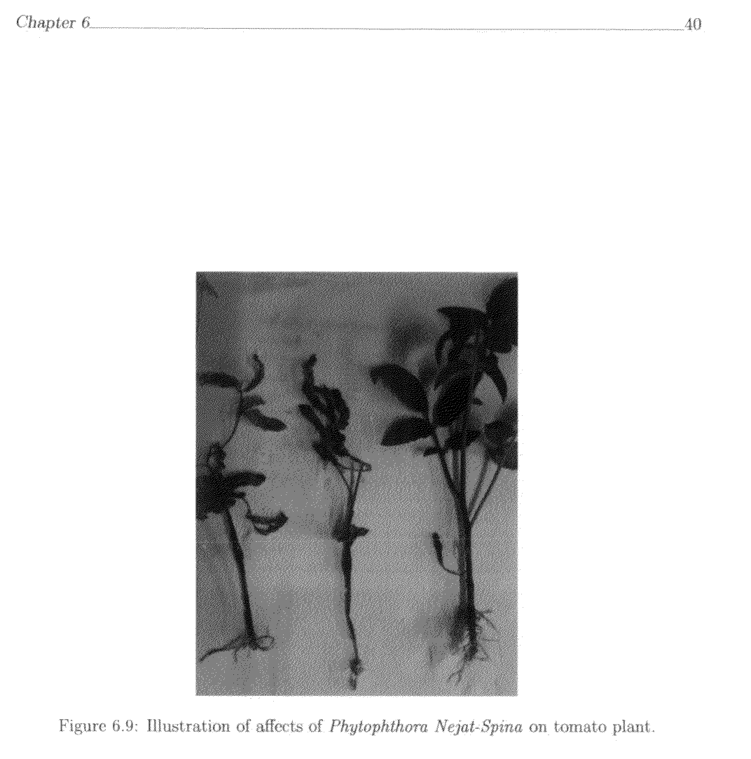

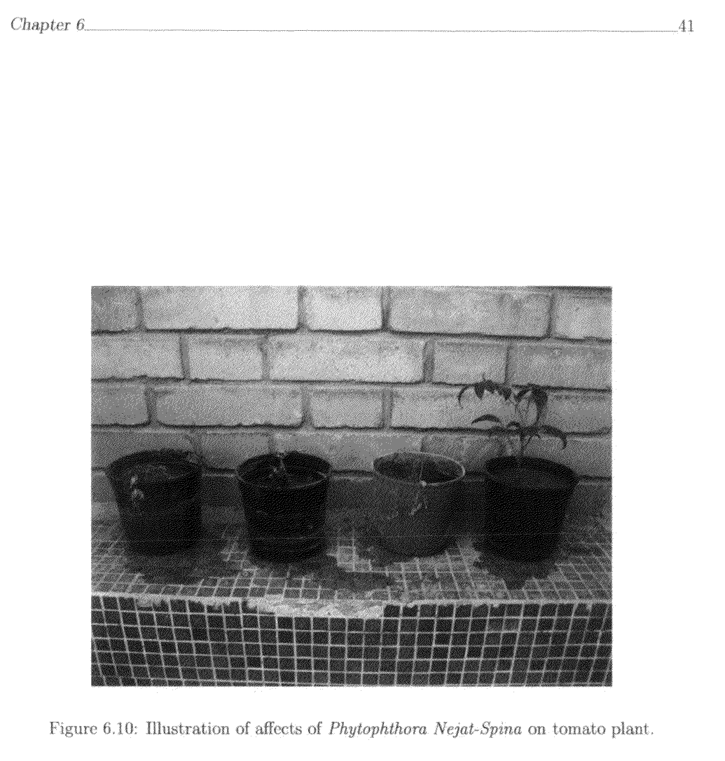

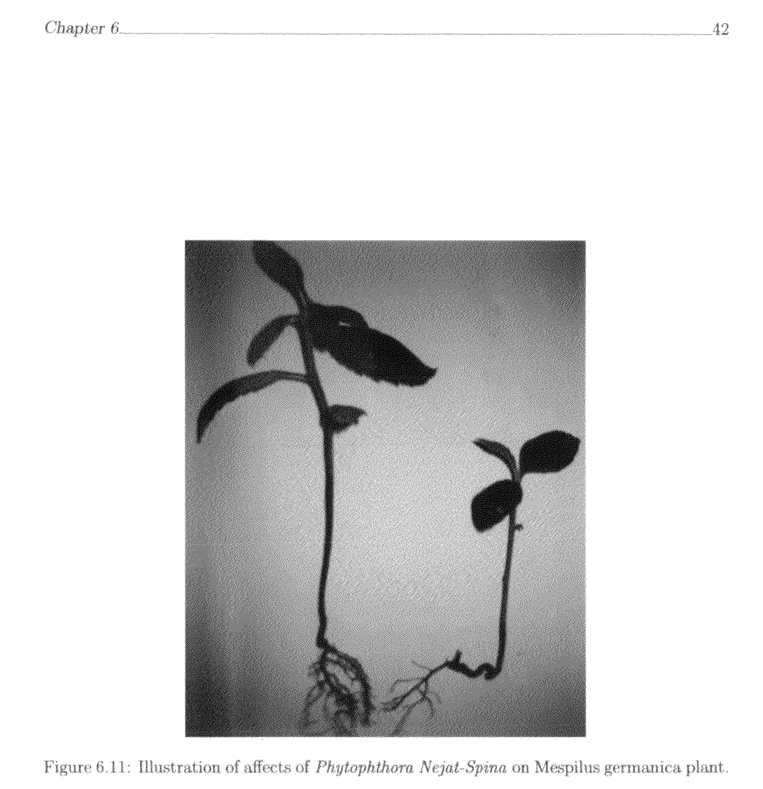

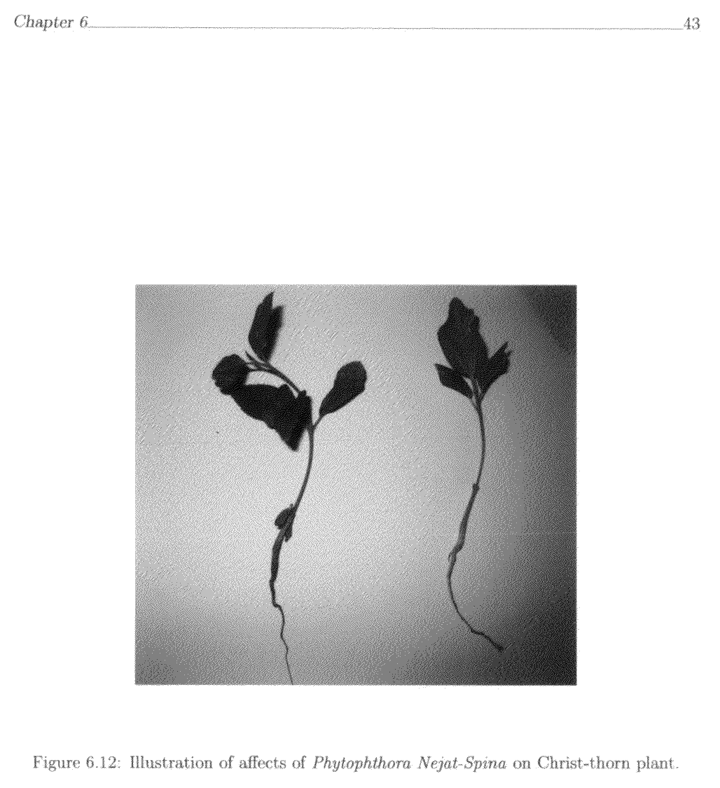

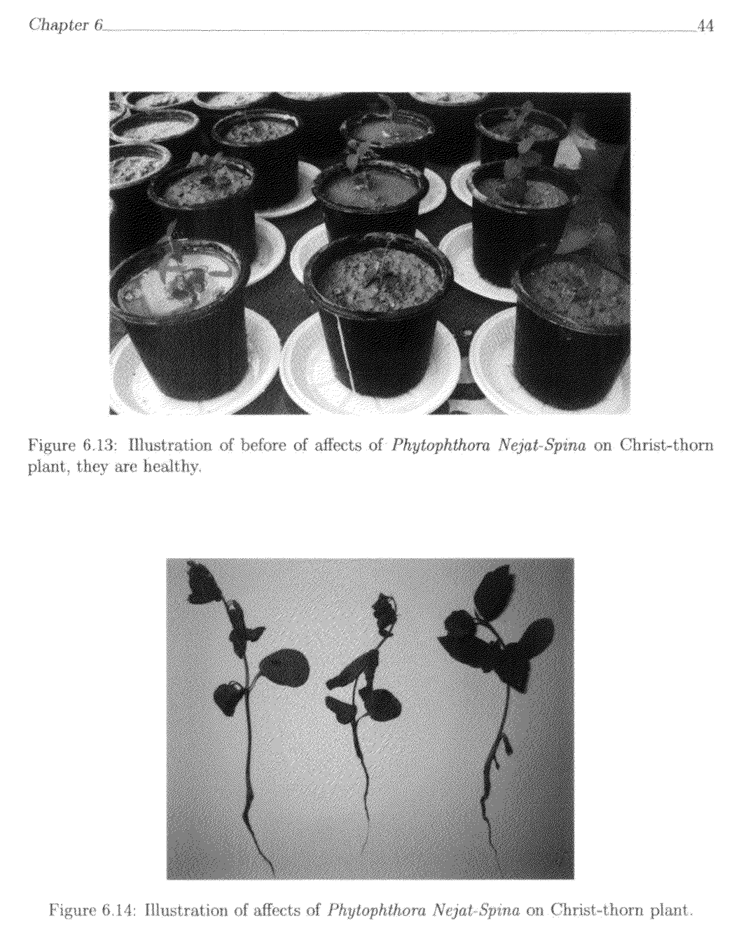

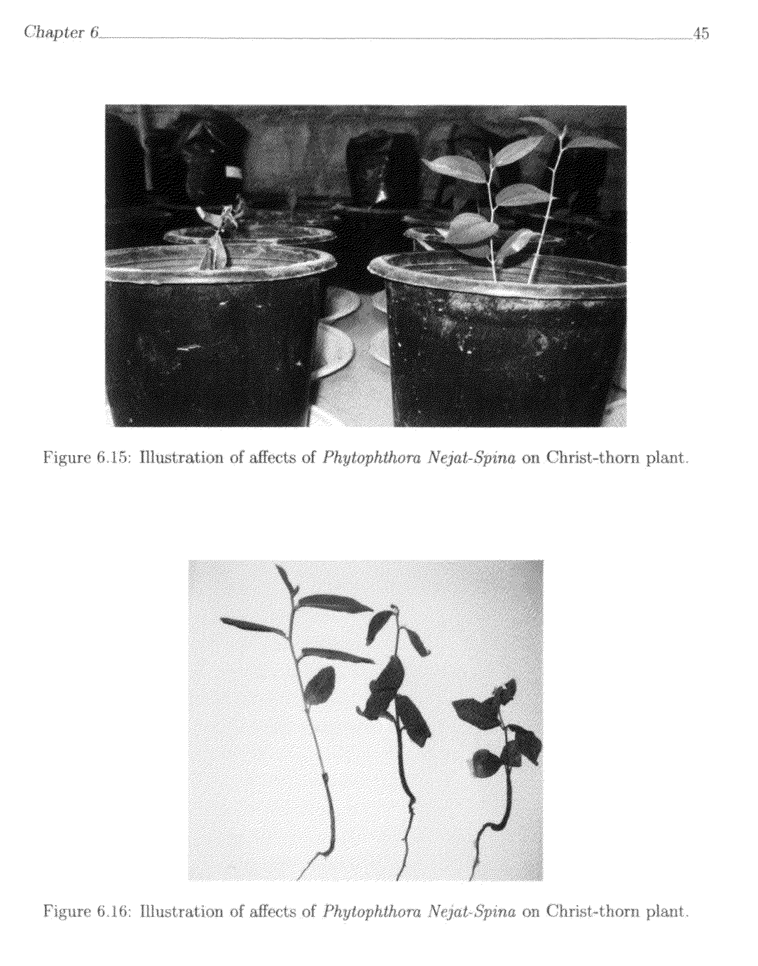

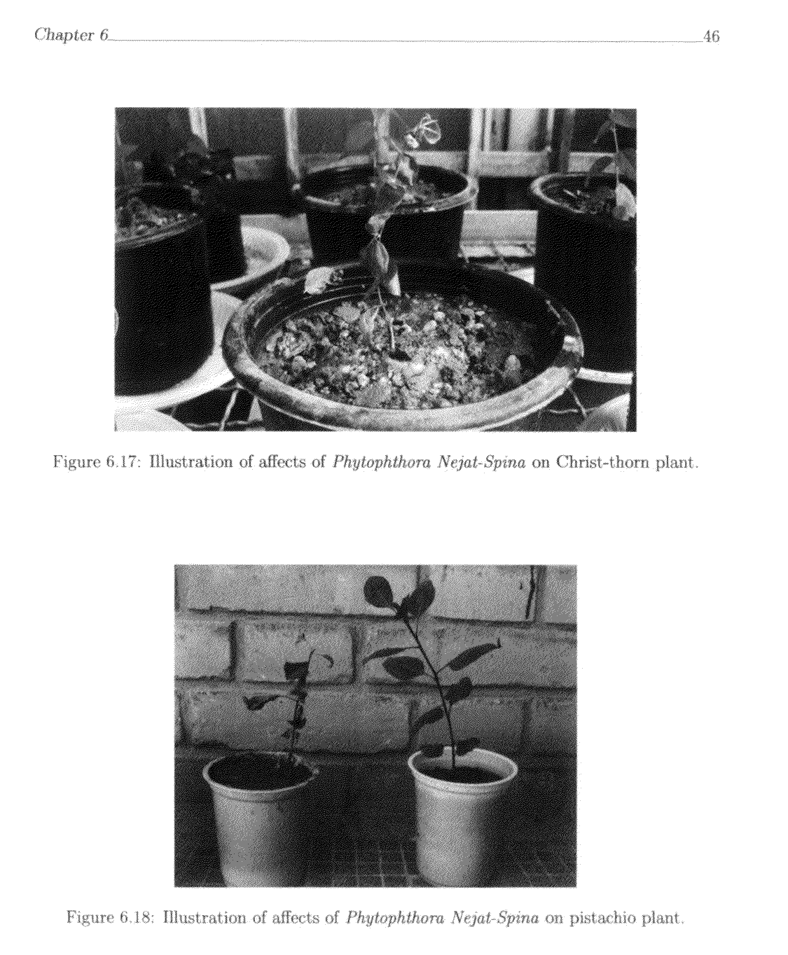

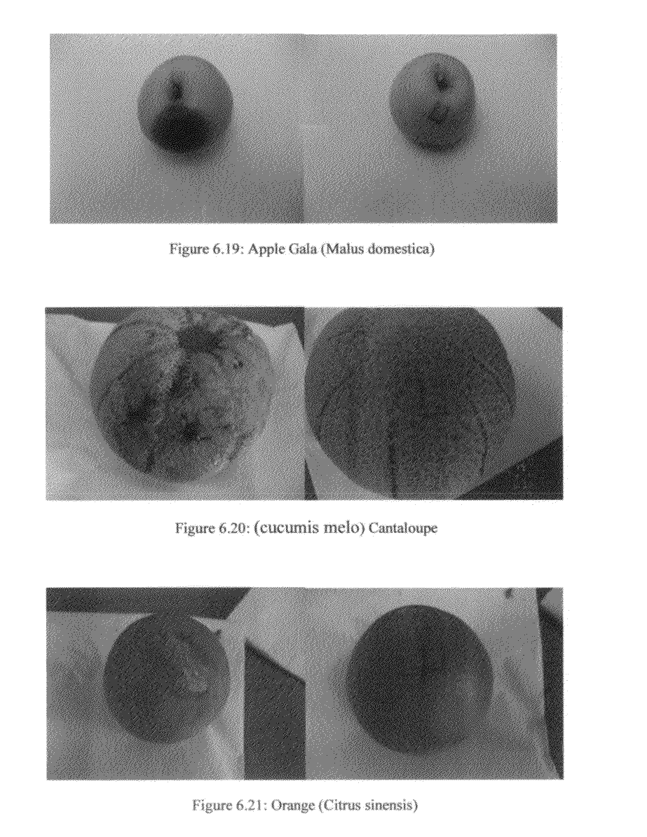

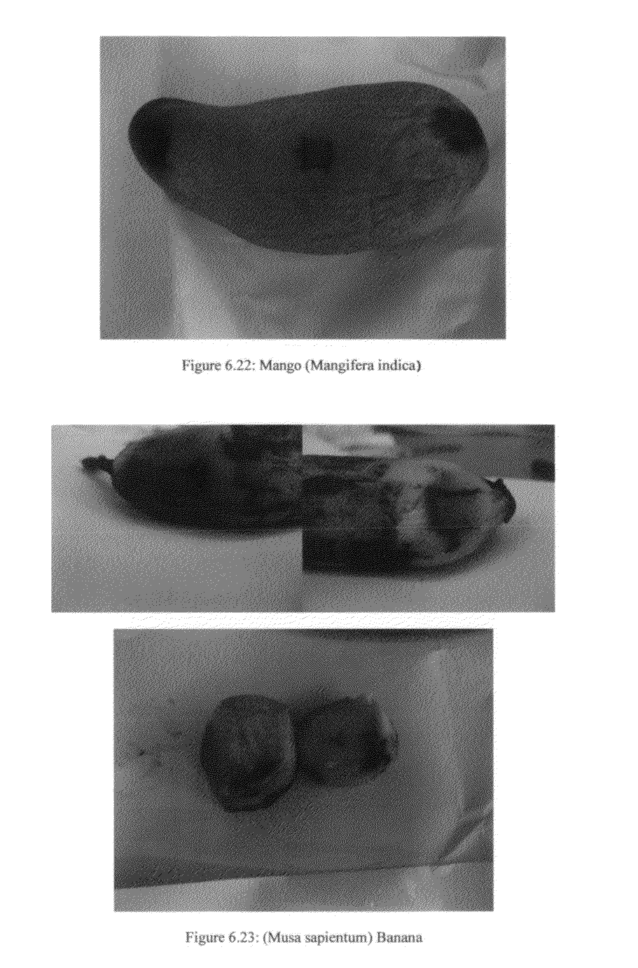

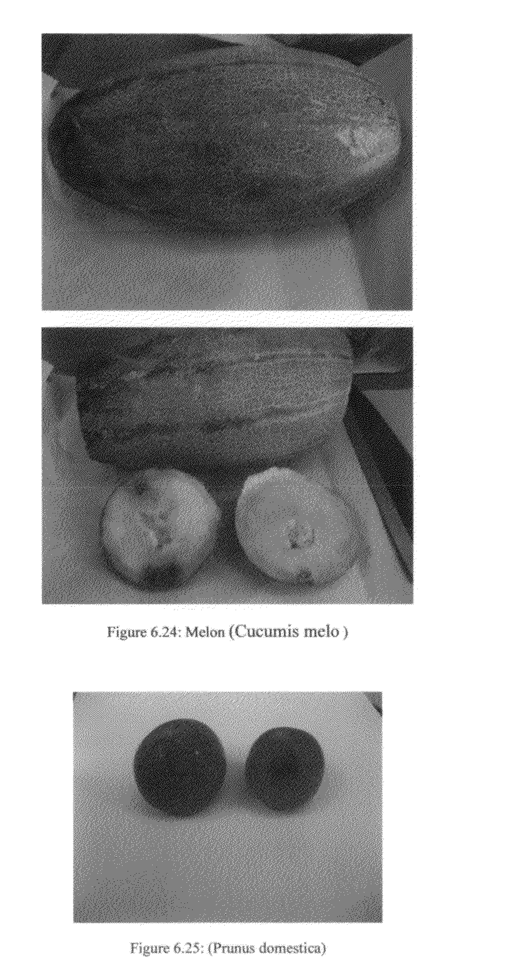

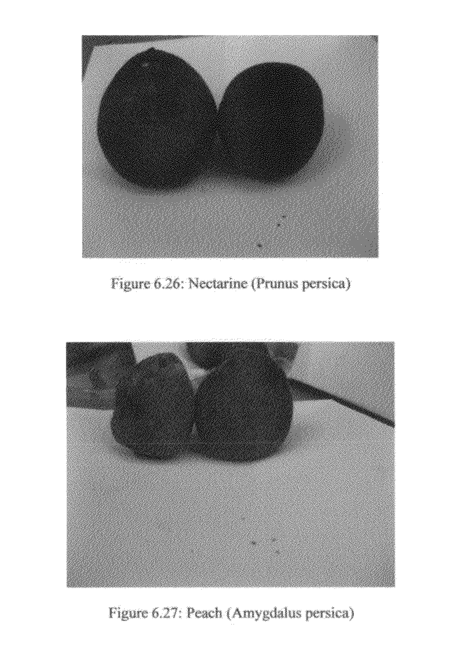

In the FIG. 2.1, the Phytophthora Nejat-Spina is compared with two other spices Phytophthora arecae and Phytophthora palmivora. In the FIG. 2.2, the Phytophthora Nejat-Spina is compared with three other spices Phytophthora Porri, Phytophthora Sojae, and Phytophthora Megasperma.

### LIST OF FIGURES

2.1 Comparing the Phytophthora Nejat-Spina with two other spices Phytophthora arecae and Phytophthora palmivora.

2.2 Comparing the Phytophthora Nejat-Spina with three other spices Phytophthora Porri, Phytophthora Sojae, and Phytophthora Megasperma.

3.1 Sporangium with medium pedicle in solid media, picture scale 400 times of real size.

3.2 Illustration of sporangium in sterile water with cannabis.

3.3 Illustration of sporangium in sterile water with cannabis.

3.4 Illustration of sporangium in sterile water with cannabis.

3.5 Illustration of Long papilla sporangium in sterile water with cannabis.

3.6 Illustration of Sporangium with medium pedicle in solid media with chlamidospore.

4.1 Illustration of Oospores with amphigynous antheridium in Carrot Agar media.

4.2 Illustration of chlamidospore with hyphal swelling at the bottom).

4.3 Illustration of Oogonia with tapered base.

4.4 Illustration of Oospor with paragynous antheridium.

4.5 Illustration of Aplerotic Oospores.

4.6 Illustration of Oospor with multi paragynous antheridium (that is very unique between Phytophthora's species).

4.7 Illustration of Oospor with paragynous antheridium.

4.8 Illustration of Oospor with multi paragynous antheridium (that is very unique between Phytophthora's species).

5.1 Illustration of terminal chlamidospore.

5.2 Illustration of terminal chlamidospore.

5.3 Illustration of terminal chlamidospore.

5.4 Illustration of chlamidospore and sporangium that observed in hem seed agar together.

5.5 Illustration of hyphal swelling.

5.6 Illustration of hyphal swelling.

5.7 Illustration of hyphal swelling.

5.8 Illustration of mycelium.

5.9 Illustration of mycelium.

5.10 Illustration of mycelium.

6.1 Illustration of cultivation samples.

6.2 Illustration of cultivation sample.

6.3 Illustration of cultivation samples, from left: 1—hemp seed agar, 2—carrot agar, 3—water agar.

6.4 Illustration of cultivation samples, from up left: CMA (CORN MEDIA AGAR), CA (CARROT AGAR), PCA (POTATO CARROT AGAR), HAS (HEMP SEED AGR), WA (WATER AGAR), PARP-CMA, PDA (POTATO AGAR).

6.5 Illustration of affects of Phytophthora Nejat-Spina on Zucchini, in above pictures the upright is a healthy Zucchini and the rest of them are affected by Phytophthora Nejat Spina.

6.6 Illustration of affects of Phytophthora Nejat-Spina on eggplant.

6.7 Illustration of affects of Phytophthora Nejat-Spina on potato.

6.8 Illustration of affects of Phytophthora Nejat-Spina on tomato plant.

6.9 Illustration of affects of Phytophthora Nejat-Spina on tomato plant.

6.10 Illustration of affects of Phytophthora Nejat-Spina on tomato plant.

6.11 Illustration of affects of Phytophthora Nejat-Spina on Mespilus germanica plant.

6.12 Illustration of affects of Phytophthora Nejat-Spina on Christ-thorn plant.

6.13 Illustration of before of affects of Phytophthora Nejat-Spina on Christ-thorn plant, they are healthy.

6.14 Illustration of affects of Phytophthora Nejat-Spina on Christ-thorn plant.

6.15 Illustration of affects of Phytophthora Nejat-Spina on Christ-thorn plant.

6.16 Illustration of affects of Phytophthora Nejat-Spina on Christ-thorn plant.

6.17 Illustration of affects of Phytophthora Nejat-Spina on Christ-thorn plant.

6.18 Illustration of affects of Phytophthora Nejat-Spina on pistachio plant.

1. Phytophthora Nejat-Spina
